(19) United States
(12) Patent Application Publication
(10) Pub. No.: US 2008/0051822 A1
(43) Pub. Date: Feb. 28, 2008

(54) METHODS AND APPARATUS FOR USE IN MEDICAL TREATMENT

(76) Inventor: Alan David Widgerow, Gauteng (ZA)

Correspondence Address: CHRISTIE, PARKER & HALE, LLP, PO BOX 7068, PASADENA, CA, 91109-7068, US

(21) Appl. No.: 11/782,625

(22) Filed: Jul. 24, 2007

Related U.S. Application Data
(63) Continuation-in-part of application No. 11/333,840, filed on Jan. 17, 2006.
(60) Provisional application No. 60/593,481, filed on Jan. 18, 2005.

(30) Foreign Application Priority Data
Jul. 25, 2006 (EP) 0601 5460.6
Aug. 16, 2005 (ZA) 2005/06174
Oct. 26, 2005 (ZA) 2005/08678

(51) Int. Cl. A61M 29/02

(52) U.S. Cl. 606/192; 604/97.01; 623/8

(57) ABSTRACT

A method of stretching the skin of a patient such as a patient who has undergone a mastectomy is described. A surgeon implants an expander under the skin of the patient and inserts a small amount of liquid to enlarge the expander. Thereafter the patient herself operates a patient controlled liquid delivery machine (“PCD”) to deliver amounts of the liquid to the expander. The PCD is arranged so that only small amounts of liquid can be introduced at each actuation thereof and that there is a delay between the times that the PCD can be actuated. Thus the expander can be increased in size in small increments which will not cause the patient pain until the expander has reached the desired size.

### CROSS-REFERENCE TO RELATED APPLICATION(S)

This application is a continuation-in-part of application Ser. No. 11/333,840 filed Jan. 17, 2006.

This application claims the benefit under 35 U.S.C. 119(a) of European Patent Office Application No. EP 06 01 5460 filed Jul. 25, 2006.

Patent application Ser. No. 11/333,840 claimed the foreign priority benefits under 35 U.S.C. 119(a), of Republic of South Africa application numbers 2005/06174 filed 16 Aug. 2005 and 2005/08678 filed 26 Oct. 2005; it also claimed, under 35 U.S.C. 119(e), the benefit of U.S. provisional application No. 60/593,481 filed on Jan. 18, 2005.

### FIELD OF INVENTION

This invention relates to methods of and apparatus for use in medical treatment.

After certain surgical procedures it is necessary to insert prostheses under the skin. However, there are often difficulties because the skin is tight and unable to stretch to accommodate the prosthesis. The procedure presently adopted to meet this problem is to insert a hollow flexible expander under the skin and to increase the volume of the expander by introducing a liquid into it and thereby stretching the skin. The procedure continues until the skin is large enough to accommodate the prosthesis there below with the patient remaining comfortable. Typical examples discussing this procedure are disclosed in WO 01/93771 and WO 96/9612518 which will be discussed more fully below. The prosthesis is then permanently inserted in position. The same procedure may be adopted to repair skin on the head, neck, hands, arms and legs where, although a prosthesis is not used, skin has to be stretched to replace damaged or removed skin. It will be understood that in certain circumstances, other tissue may also have to be stretched/expanded and the term “skin” as used herein is intended to cover such tissue, unless clear from the context.

In known procedures, the expander is inserted under the skin and a permanently attached flexible tube is usually buried under the skin. A sealed receiver unit/valve, which is covered by a membrane, is connected to the distal end of the flexible tube and buried beneath the skin or may be integrated into the expander. The patient now visits a medical practitioner, normally a surgeon, who introduces the liquid into the expander through the valve beneath the skin thereby enlarging it to stretch the skin. This the medical practitioner does by using a fine needle attached to a syringe to pierce the skin and valve membrane and to feed liquid into the receiver unit valve from whence it passes via the flexible tube into the expander. It will be understood that the stretching of the skin must take place in small increments. If the skin is stretched to too great an extent, the patient will suffer discomfort or pain, and in extreme cases the scar tissue may tear. This limits the amount of expansion is of the expander during each visit to the medical practitioner who has to expend considerable care and expertise in feeding liquid to the expander. Consequently the treatments required which normally take place on a weekly basis, are numerous, and, for example, in the case of stretching skin after a mastectomy, the procedures may extend over a period of as long as three months. Furthermore, of course, the procedure is expensive and inconvenient requiring the patient to visit the practitioner (usually necessitating travel, waiting in the reception room etc). In addition until the reconstructive surgery takes place the patient will suffer from abnormal appearance due to the expansion as aforesaid. There will also be increased risk of complications where the expansion takes place over an extended period of time. For all these reasons it is desirable for the expansion period to be decreased which would contribute to patient convenience, decreased costs and improved acceptance of this technique.

### SUMMARY OF THE INVENTION

According to one aspect of the invention there is provided apparatus for use in expanding skin, comprising an inflatable expander that is capable of being introduced under the skin of a patient which is required to be stretched, a flexible tube leading from the expander and being capable of passing through the skin of the patient, an interconnector attached to the tube outside the body of the patient, and a liquid delivery machine connectable to the interconnector, the machine including a pump having a pump chamber, an inlet port leading to the pump chamber, flow control means for controlling flow into the pump chamber, an outlet port from the pump chamber leading to a non-return valve characterised in that the liquid delivery machine comprises a cylinder, a piston within the cylinder movable from an initial position in which the volume of the cylinder is at a maximum and a depressed position in which the volume of the cylinder is at a minimum, manually operable operating means which is manually operable to move the piston from the initial position to the depressed position, and spring means for biasing the piston to the initial position, and in that the strength of the spring means and the effect of the flow control means is such that a substantial time, for example of the order of 5 to 10 minutes passes before the piston is moved from the depressed position to the initial position.

Normally a patient using this apparatus will not deliver too much liquid by means of the liquid delivery machine because discomfort and pain will prevent such excessive delivery. Preferably, however, the liquid delivery machine incorporates means for ensuring that only limited amounts of liquid such as 1 to 2 ml can be dispensed thereby during a specified time period.

Preferably connector means are provided between the interconnector and the dispensing end of the liquid delivery machine, which are easily cleanable so that the patient may maintain the apparatus sterile. A covering is normally provided for the interconnector, so that this is protected when not in use. Similarly, a covering member may be provided for the dispensing end of the liquid delivery machine.

An embodiment of the invention will now be described by way of example, with reference to the accompanying drawings.

### BRIEF DESCRIPTION OF THE DRAWINGS

In the drawings:—

FIG. 1 is a perspective view of an expander of the invention, incorporating a trocar,

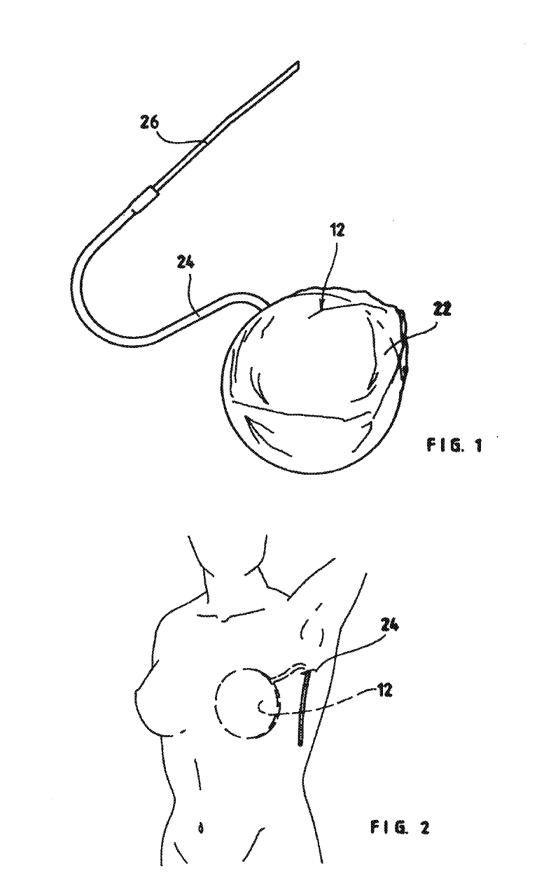

FIG. 2 is a diagrammatic view showing the position of the expander applied to a patient who has had a mastectomy,

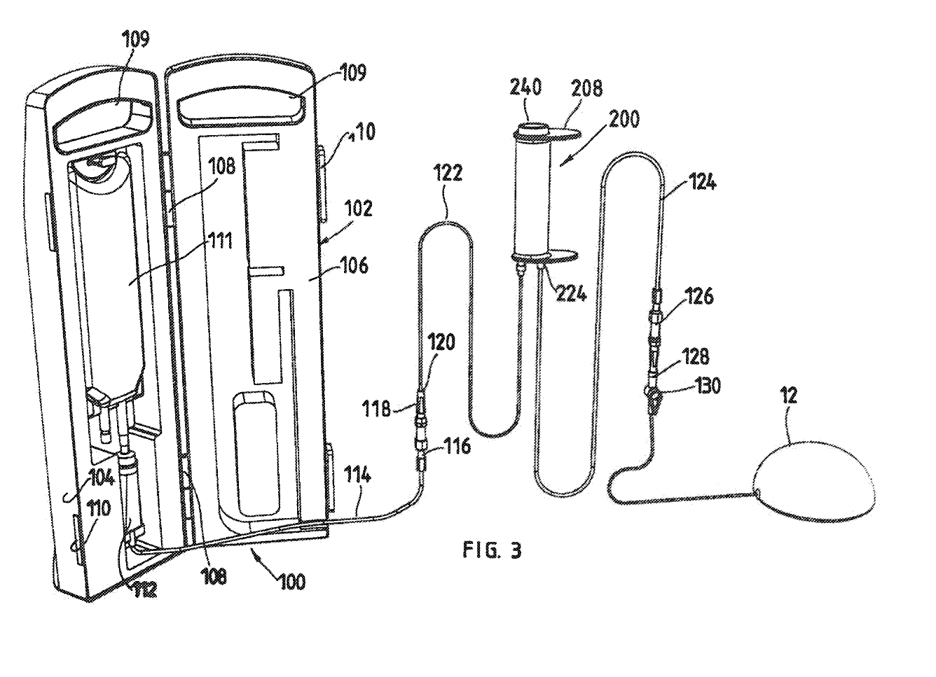

FIG. 3 is a view of a PCD machine with the container in the open position,

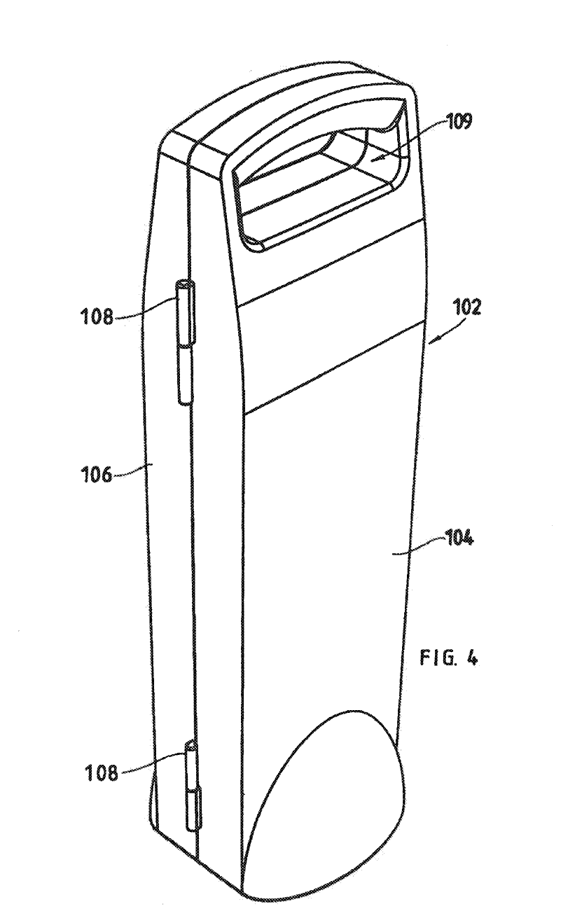

FIG. 4 shows the container closed,

FIG. 5 is a section on line 5-5 of FIG. 3, and

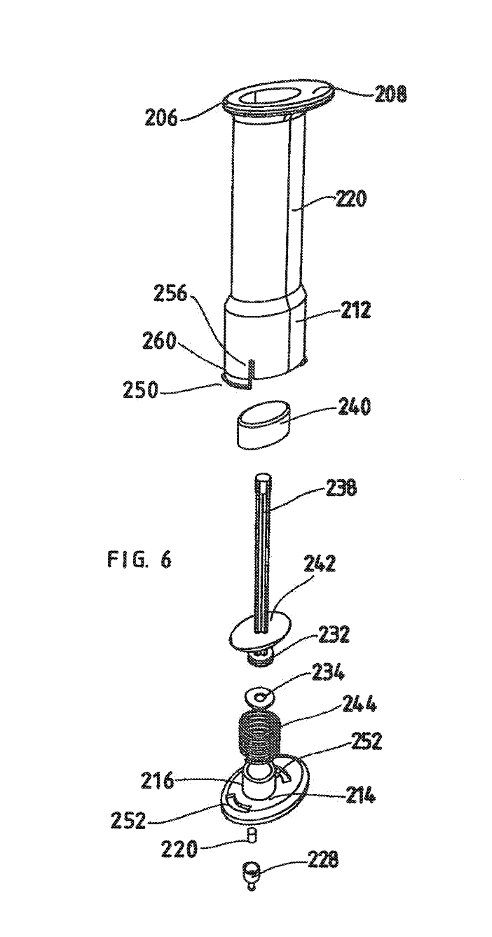

FIG. 6 is an exploded view of the PCD machine.

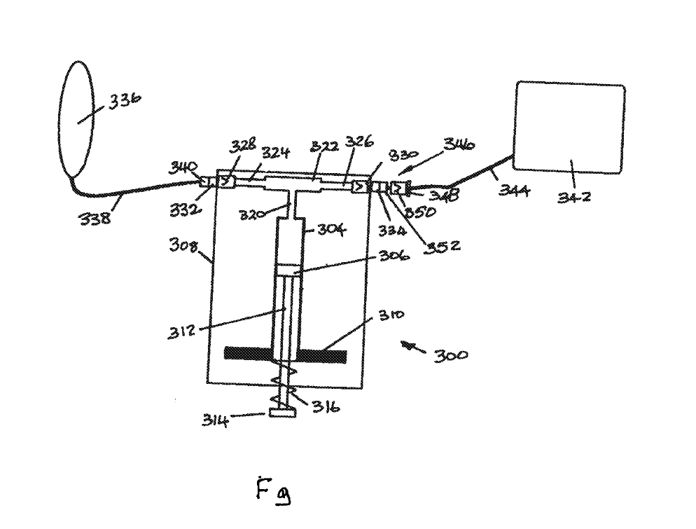

### BRIEF DESCRIPTION OF THE INVENTION

Referring now to the drawings, apparatus 10 of the invention comprises an expander 12, an intermediate connector set 14 and a patient controlled liquid delivery machine (“PCD machine”) 100 tube described more fully below together with a connector pipe 18.

The expander 12 comprises a hollow plastic member having a reasonably rigid circular base 20 and a collapsible part spherical top part 22 formed of flexible and expandable plastic material. A permanently connected flexible silicone tube 24 is connected to the expander 12, adjacent to the base 20. At its free end, the tube 24 is connected to a trocar 26. When a liquid is introduced into the expander 12, as will be described below, the volume of the expander increases and thus stretches the skin covering the expander 12.

The PCD machine 100 has a housing 102 comprising two members 104 and 106 which are both plastics mouldings and which are connected together by hinge members 108. The interior of the members 104 and 106 are suitably shaped to provide recesses for the parts that will be described below so that when closed there will be a compact unit which a patient can carry and store as desired. A transverse opening 109 passes through the upper ends of the members 104 and 106 to form a handle to facilitate carrying the housing 102. Suitable closure means 110 are provided to hold the members together when the housing is closed.

Within one member 104 is carried a bag 111 containing saline and connected to a suitable filter mechanism 112 as is well known in the medical profession. The mechanism 112 leads to a narrow tube 114 which terminates in a one way valve with an antibacterial filter 116 and having a male luer connector 118. A female luer connector 120 is connected by a silicon tube 122 to the inlet duct of a pump 200 (to be described below) the outlet duct of which leads via a silicon tube 124 to another one way valve 126 incorporating an antibacterial filter and having a male luer connector 128 which may connect to junction 130 incorporating a lever operated control valve 132 that in turn is connected to the tube leading to the expander 12. This junction 130 serves as an interconnector to the tube 24.

The pump 200 comprises an enlarged hollow gripping part 202 that is oval in section. It has an open top end 204 which is flanged and the flange 206 has an enlarged part 208 at one of its smaller sides. At its lower end 210 is an enlarged portion 212 mounted on a bottom cross plate 214. On the cross plate 214 there is centrally mounted a short cylinder 216 forming a pump chamber as will become apparent. At its lower end the cylinder 216 has an inlet port 218 passing through the cross plate 214 and being connected to a short connector tube 220 and an outlet port 222 also passing through the cross plate 214 and being connected to an outlet tube 224. The inlet tube 220 carries a union 226 within which is a control valve or flow limiter 228.

Internally at the juncture of the gripping part 202 and the enlarged portion 212 is a shoulder 230 facing the cross plate 214. A piston member 232 is mounted in the cylinder 216 being sealed thereto by a suitable cap seal 234 with an enlarged ring surround 236. The piston member 232 is movable from an initial position in which the volume of the cylinder 216 is at a maximum, to a depressed position in which the volume of the cylinder is at a minimum. The piston member 232 is carried at the lower end of an elongated rod 238 that is cruciform in section. A smooth sided operating button 240 is provided at the upper end of the rod 238 and this projects slightly above the flange 206. A guide member 242 is mounted on the rod 238 within the enlarged portion and is slidably mounted therein from an upper position in which it butts against the shoulder 230 and a lower position determined by the engagement of the piston member 232 against the part of the cross plate 214 forming the bottom of the cylinder 216. By depressing the button 240 the user moves the piston from initial position to the depressed position. A spring 244 biasses the guide member 242 towards and against the shoulder 230 and in moving to this position, the guide member moves the button from the depressed position to the initial position. The dimensions of the cylinder 216 and the stroke of the piston member 232 is such that on each operation about 2 ml liquid is pumped. The flow limiter 228 determines the speed that liquid can enter the cylinder 216 below the piston member 232, it being noted that significant amounts of liquid cannot be drawn in through the outlet 224 and tube 124 because of the provision of the one way valve 126. This coupled with the strength of the spring 244 determines the time taken for the piston member to move to the initial position so that the button 242 will be in position to be depressed. The device is pre-set so that cylinder 216 will be refilled only after a predetermined and relatively substantial time which may conveniently be between five and ten minutes. Thus only limited amounts of liquid can be dispersed during any specified time period.

The gripping part 202 is connected to the cross plate 214 by means of extension pieces 250 that pass through arcuate slots 252 in the cross plate 214 and have outwardly projecting flange portions 254 that lie under the cross plate 214. The enlarged portion 212 has two pairs of vertical slots 256 and 258 which form resilient arms 260 and 262 that carry the extension pieces 250 and that can be pivoted inwardly to permit the flange portions 254 to pass through the slots 252 and when released the arms 260 and 262 move outwardly so that the flange portions lie under the cross plate 214 to hold the gripping part thereto. Thus the pump 200 can be disassembled for cleaning and sterilizing and for adjusting the volume of liquid expressed.

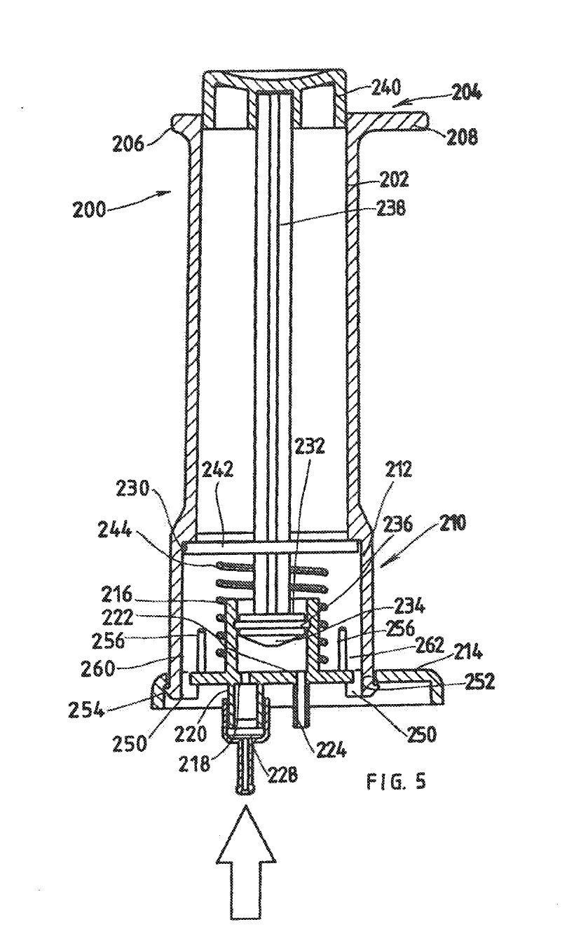

In use, after a mastectomy, the expander 12, which is in the collapsed condition, is surgically fitted under the skin retropectorally on the mastectomy side in the normal fashion (see FIG. 4). By means of the trocar 26 an opening is made in the skin, preferably below the arm pit, so that the tube 24 is exposed to the external atmosphere. The trocar 26 is now removed by cutting the end part of the tube 24. The junction 130 is connected to the free end of the tube 24. An amount of liquid is introduced into the expander by the surgeon. The control valve 132 is closed, and if desired a cap or other cover is placed over the junction. The exposed part of the tube 24 is bound to the patient in a way to protect this item and to cause the patient minimum discomfort.

After a period of about 1-5 days after the implant procedure (or sooner in some cases when the surgeon believes applicable) the patient may now commence expansion.

The patient will now use the housing 100 and will add a fresh bag of saline 111. By means of the one way valve 116 the Luer connectors 118 and 120 the bag is connected to the pump 200. The patient will prime the pump by pressing the operating button 240 a few occasions until she sees that liquid is discharged from the outlet filter 126. The patient then fits the connector 128 to the control valve. By depressing the button 240 at this stage the patient forces about 2 ml liquid into the expander. This may be adequate at this stage. The patient when comfortable may wish to depress the button 240 again to expand the expander. However, as mentioned above the pump remains inactive for ten minutes (thus the patient will not overfill the expander). The patient can then insert further amounts of saline into the expander as desired but ensuring that there is not so much inserted as to cause discomfort. By increasing the expander size with the small amounts of liquid at reasonably spaced intervals the skin will stretch without undue pain or discomfort.

Because the button is smooth and projects only a small amount from the top of the gripping part the patient cannot hold this and pull on it to supplement the effect of the spring 244 to cause the cylinder to be recharged more quickly than the time that has been set. By depressing the button 240, the patient ensures that a preset amount of liquid is introduced into the expander 12. The PCD machine 100 prevents excessive introduction of liquid into the expander 12 during each procedure. However, the procedure can be repeated multiple times a day so that the skin is stretched to a small extent after each procedure. After each stretching operation the skin relaxes, so that during the next stretching operation the skin will be ready to stretch further and pain will be reduced.

The patient may apply topical creams to the skin being stretched to facilitate the process of stretching of the skin and to ease any pain involved. It is desirable that the surgeon or the medical practitioner monitors the process once or twice weekly which may or may not involve visits to the surgeon or medical practitioner. The process is continued until the breast is slightly over expanded which should be determined by the surgeon and is left as such until the next stage is scheduled. The expander is then removed and the normal routine of the breast reconstruction is continued.

We have found that it is relatively easy to instruct the patient how to operate and refill the PCD and how to keep the various parts clean and sterile. It is also easy to disassemble the PCD machine for cleaning and sterilization.

We have found that because the expansion takes place regularly with very small increments, it is possible to complete the full expansion of the expander in a very short period of time such as typically three weeks. Thus the reconstruction time can be decreased by about two months or more. Further as the visits to the medical practitioner can be reduced significantly the cost of the procedure will also be considerably reduced.

The invention is not limited to the precise details hereinbefore described and illustrated. For example the same procedure may be adopted for any case of tissue expansion e.g. to repair skin on the head, neck, hands, arms and legs where, although a prosthesis is not used, skin has to be stretched to replace damaged or removed skin. The shape of the expander would vary according to the needs of the reconstruction. The expander may vary as will be appreciated by those skilled in this art. The use of procedure on the skin on the head has a benefit in that hair will grow through this skin which usually does not occur where skin is taken from another part of the patients body. The amount of liquid provided on each operation of the PCD machine may vary as desired although this usually no more than 1-5 millilitres of liquid being introduced. A non-return valve may be provided at the outlet tube. Other liquids may be used to enlarge the expander.

In a further modification pressure reading monitors may be attached to the expander tube enabling the monitoring of pressure transmitted within the expander and on to the overlying skin.

Future scenarios are envisaged where teletype monitoring from home is possible, whereby photo or video documentation of the status of the expansion is transmitted to the surgical control centre for monitoring by medical personnel, e.g. a surgeon, a doctor or specially trained nurse.

What is claimed is:

1) Apparatus for use in expanding skin, comprising
an inflatable expander that is capable of being introduced under the skin of a patient which is required to be stretched,
a flexible tube leading from the expander and being capable of passing through the skin of the patient,
an interconnector attached to the tube outside the body of the patient, and
a liquid delivery machine connectable to the interconnector, the machine including a pump having
a pump chamber,
an inlet port leading to the pump chamber, and
an outlet port leading from the pump chamber leading to a non-return valve and thence to the expander,
wherein the liquid delivery machine comprises
a cylinder forming the pump chamber,
a piston within the cylinder movable from an initial position in which the volume of the cylinder is at a maximum and a depressed position in which the volume of the cylinder is at a minimum,
manually operable operating means which is manually operable to move the piston from the initial position to the depressed position, and
spring means for biasing the piston to the initial position,
and wherein the strength of the spring means and the effect of the flow control means is such that a substantial time passes before the piston is moved from the depressed position to the initial position.

2) Apparatus as claimed in claim 1 further comprising flow control means for controlling flow into the pump chamber wherein the effect of the flow control means combines with the strength of the spring so that a substantial time passes before the piston is moved from the depressed position to the initial position.

3) Apparatus as claimed in claim 1 wherein the said substantial amount of time is between five to ten minutes.

4) Apparatus as claimed in claim 1 wherein the liquid delivery-machine incorporates means for ensuring that only a limited amount of liquid can be dispensed thereby during a specified time period.

5) Apparatus as claimed in claim 4 wherein the said limited amount of liquid comprises 2 ml liquid.

6) Apparatus as claimed in claim 1 wherein the operating means comprises a button which is connected to the piston by a connecting device.

7) Apparatus as claimed in claim 6 wherein the cylinder is shaped to be able to be gripped manually and has the button protruding from one end thereof so that when the cylinder is gripped a patient can depress the button by means of the patient's thumb.

8) Apparatus as claimed in claim 7 wherein the button projects only a small distance from the cylinder and is smooth-sided so that the patient cannot grip the button to speed the movement of the piston to the initial position.

9) Apparatus as claimed in claim 1 wherein a casing is provided for all the parts which will enable the patient to move the device conveniently to any suitable place for use.

10) Apparatus as claimed in claim 1 further comprising a housing containing the pump, wherein the housing has inlet and outlets controlled by one way valves leading to a chamber connected to the cylinder.
